(19) United States
(12) Patent Application Publication
(10) Pub. No.: US 2024/0203575 A1
(43) Pub. Date: Jun. 20, 2024

(54) METHOD FOR COMPUTING A PROCESS DESCRIPTION OF AN AUTOMATED OR SEMI-AUTOMATED MEDICAL DEVICE REPROCESSING PROCEDURE

(71) Applicant: Olympus Winter & Ibe GmbH, Hamburg (DE)

(72) Inventors: Sascha Jaskola, Hamburg (DE); Stefan Schroeder, Hamburg (DE)

(73) Assignee: Olympus Winter & Ibe GmbH, Hamburg (DE)

(21) Appl. No.: 18/539,011

(22) Filed: Dec. 13, 2023

Related U.S. Application Data
(60) Provisional application No. 63/432,773, filed on Dec. 15, 2022.

(51) Int. Cl. G16H 40/40

(52) U.S. Cl. CPC G16H 40/40

(57) ABSTRACT

A method for computing a process description of an automated or semi-automated medical device reprocessing procedure. The process description including a plurality of procedural steps including at least one of: manual pre-cleaning, automated chemical cleaning and disinfection, thermal sterilization, and drying. The method including: collecting log data of completed reprocessing procedures, the log data including, for a plurality of procedural steps: the type of the respective procedural step, an identifier of a medical device undergoing the procedure, a start time of the procedural step, a duration or an end time of the procedural step, and a success indicator of the procedural step; feeding the collected log data to a data analyzing engine, and computing, a process description of the semi-automated or automated reprocessing procedure. Wherein the process description includes a sequence of procedural steps necessary for the reprocessing procedure, and the duration of each of the procedural steps.

### CROSS-REFERENCE TO RELATED APPLICATION

The present application is based upon and claims the benefit of priority from U.S. Provisional Application No. 63/432,773 filed on Dec. 15, 2022, the entire contents of which is incorporated herein by reference.

### BACKGROUND

### Field

The present disclosure relates to reprocessing of medical devices. More specifically, the present disclosure relates to a method and computer program product for computing the process description of an automated or semi-automated medical device reprocessing procedure.

### Prior Art

In modern medicine, a large variety of different medical instruments and devices is routinely used. Such instruments and devices range from simple instruments, for example, scalpels and forceps, to sophisticated devices, such as rigid or flexible video endoscopes. While some instruments are intended for single use only and must be discarded after use, many modern devices are reusable and must therefore undergo careful reprocessing after each use. Reprocessing of medical instruments or devices includes steps such as manual precleaning, automated chemical cleaning and disinfection, thermal sterilization, drying, and the like. In hospitals and larger medical offices, dedicated reprocessing departments are responsible for such reprocessing.

Due to medical safety regulations, reprocessing instructions for each type of instrument or device must be available. Such reprocessing instructions are usually prepared manually and retained in a paper or electronic repository. As a further requirement each reprocessing procedure applied to a specific instrument or device needs to be documented. Such documentation is usually prepared and stored electronically.

It has been observed in the field, that reprocessing instructions are not always fully complied with. In many cases, minor deviations can be observed, often resulting from constraints which have not been considered during preparation of the reprocessing instructions. Such constraints can for example be a time required for transporting an instrument or device between different reprocessing equipment. But also other modifications or optimizations of reprocessing procedures have been observed, which did not fully reflect in the retained reprocessing instructions.

It is becoming popular for hospitals and larger medical offices to employ digital planning tools for scheduling procedures such as examinations or interventions. Such planning tools sometimes also provide for scheduling the reprocessing of instruments and devices used in such procedures, so that fully reprocessed instruments and devices are always available for any scheduled procedures.

When such a sophisticated planning tool is introduced to a new office or hospital, it is necessary to teach in all the reprocessing instructions currently in force in the facility. Due to the high number of different instruments and devices used, usually takes a significant effort to do so.

### SUMMARY

It is therefore an object of the present disclosure to provide improved methods for computing process descriptions of automated or semi-automated medical device reprocessing procedures so that they can be used in the aforementioned planning tools.

The present disclosure provides a method for computing the process description of an automated or semi-automated medical device reprocessing procedure, the process description comprising a plurality of procedural steps including at least one of: manual pre-cleaning, automated chemical cleaning and disinfection, thermal sterilization, drying, and storage; the method comprising: collecting log data of completed reprocessing procedures, the log data including, for a plurality of procedural steps: the type of the respective procedural step, an identifier of a medical device undergoing the procedure, a start time of the procedural step, a duration or an end time of the procedural step, and a success indicator of the procedural step; feeding the collected log data to a data analyzing engine, and computing, through the data analyzing engine, a process description of the automated or semi-automated reprocessing procedure, wherein the process description comprises a sequence of procedural steps necessary for the reprocessing procedure, and the duration of each of the procedural steps.

Through the automatic data analysis according to this disclosure, the computed process description can exactly match the reprocessing procedure as it is actually applied. Scheduling problems arising from differences between documented and executed reprocessing procedures can therefore be avoided.

The method may further comprise computing, through the data analyzing engine, for at least one procedural step, the process constraints selected from a minimum duration and the maximum duration of the procedural step. Such constraints can be determined by applying statistical methods to the log data like calculation of an average duration, a standard deviation of the duration, a confidence interval of the duration of the respective procedural step, or the like.

Similar to the duration of the procedural step, other parameters may also be derived from the log data. Such other parameters may include applied temperatures, dosage of cleaning or disinfection chemicals, or the like. The other parameters to be stored in the log data may be an operator ID of an operator involved in the procedural step, for example, an operator performing manual precleaning, or an operator involved in loading or unloading an endoscope reprocessing machine.

The method may further comprise identifying, through the data analyzing engine, undocumented procedural steps of the reprocessing procedure, and including such undocumented procedural steps in the process description. Undocumented procedural steps may for example be transport steps, where a medical device undergoing the reprocessing procedure is moved from one reprocessing station or equipment to another reprocessing station or equipment. Such undocumented procedural steps can for example be identified through gaps between the end time of one procedural step and the start time of a following procedural step. By including such undocumented procedural steps into the process description, those steps can automatically be accounted for when scheduling the reprocessing procedures.

The method may comprise identifying, through the data analyzing engine, fallback loops of the reprocessing procedure. Fallback loops may apply in cases where certain procedural steps have not been successfully completed. Depending on the kind of procedural step, it may be necessary to repeat the same procedural step, or loop back to a previous procedural step, which needs to be repeated as well.

The method may further comprise identifying, through the data analyzing engine, variations in one procedural step correlating with variations and/or absence of the preceding procedural step. It may thus be possible to automatically recognize that a certain procedural step can be modified in order to compensate for shortcomings of preceding procedural steps, or that a certain procedural step is only required after the procedural steps have or have not been completed.

The log data may comprise log data of reprocessing procedures applied to different medical devices, and the method may comprise computing, through the data analyzing engine, separate process descriptions for each type of medical device. It is thus possible to bulk-feed a complete body of log data from a facility, and to automatically compute individual process descriptions for reprocessing all kinds of medical devices and instruments used in that facility.

The log data may include log data of reprocessing procedures applied at different locations, such as, different facilities, and the log data may include data identifying the location at which the respective reprocessing procedure has been applied. It is thus possible to process log data from multiple sites, facilities, or locations, in one run. In this case, the method may include identifying, through the data analyzing engine, differences between corresponding procedural steps at different locations.

Such differences may arise from different equipment used in different facilities, from different facility layouts resulting in different transport times, or from differences in environmental conditions such as temperature, humidity, or the like.

The present disclosure can further provide a computer program product comprising machine readable instructions for executing by a computer, configured to cause the computer to execute a method according to the above description. For effects and advantages, reference is made to the aforementioned effects and advantages.

### BRIEF DESCRIPTION OF THE DRAWINGS

The disclosure is further described in more detail with regard to exemplary embodiments and drawings. Such embodiments and drawings are only provided for better understanding the concept of the disclosure, without limiting the scope of protection, which is defined by the appended claims.

In the drawings:

FIG. 1 illustrates a provisioning system for endoscopes, and

FIG. 2 illustrates an algorithm for computing a process description.

### DETAIL DESCRIPTION

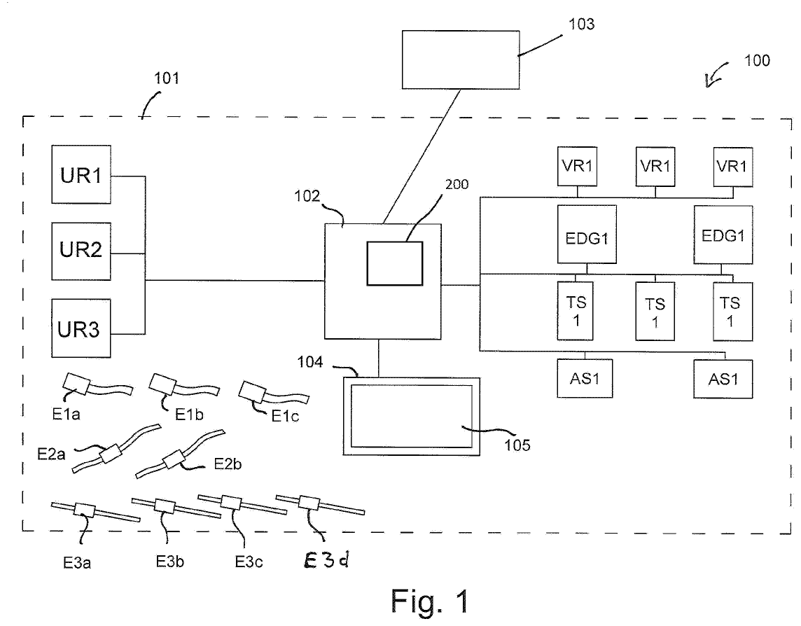

FIG. 1 shows a provisioning system 100 for endoscopes in an exemplary application environment 101, which may be an endoscopy office or an endoscopy department of a hospital.

The application environment 101 comprises several examination rooms UR1, UR2, UR3. The actual number of examination rooms may vary. The term “examination room” does not exclude interventional procedures being applied in such rooms, such as biopsies or the like.

The application environment 101 further comprises a reprocessing station for reprocessing used endoscopes comprising manual precleaning stations VR1, VR2, VR3, and a scope reprocessing devices EDG1, EDG2, drying cabinets TS1, TS2, TS3, and storing compartments AS1, AS2, the number of the respective elements again is arbitrary and only serves as an example.

For performing endoscopic examinations in the examination rooms UR1, UR2, UR3, several endoscopes of different types are kept available. For example, endoscopes E1a, E1b, E1c of a first type E1, endoscopes E2a, E2b of a second type E2, and endoscopes E3a, E3b, E3c, E3d of a third type E3 may be used in the application environment 101. Of course, other type endoscopes may also be available.

The provisioning system 100 comprises a data processing unit 102, which is configured to receive and store information regarding plan and/or current examination procedures and reprocessing procedures, and, if applicable, the present location and status of prospective endoscopes. The data processing unit 102 comprises hardware, such as a hardware processor, controller, CPU, computer etc., operating on software instructions or a hardware circuit.

Information regarding scheduled procedures can be provided to the data processing unit 102 from the hospital or office administration system 103. This can for example be done using a standardized interface, e.g., using the DICOM standard or the HL7 standard. Such information may contain the kind of the procedure, the scheduled start of the procedure, and the examination room, in which the procedure is to be performed.

Information regarding pending reprocessing procedures can be provided by the endoscope reprocessing devices EDG1, EDG2. For determining the location of the respective endoscopes, sensors can be provided in the examination rooms UR1, UR2, UR3 and at the reprocessing stations, which can detect identification features of endoscopes present in the examination room or at the reprocessing station.

Information regarding the current status of individual endoscopes can be derived from the above-mentioned information. For example, if an endoscope is present in an examination room, as a current status it may be assumed that the endoscope is currently in use. If, instead, the endoscope is present at a manual preeleaning station, it may be assumed that the endoscope is currently undergoing manual precleaning. It may additionally be detected since when the endoscope is undergoing the precleaning.

The endoscope reprocessing devices usually also provide information showing the kind and progress of a selected reprocessing program, which also indicates the status of the endoscopes presently being reprocessed in the respective endoscope reprocessing device.

From such received data, the provisioning system 100 can determine, if enough suitable endoscopes are available for the planned examination procedures. Therefore, each of the planned procedures can be assigned one or more compatible endoscope types, with which procedure can be performed. At the same time, for each endoscope type, the number of endoscopes presently available can be determined. Herein, an endoscope is considered available, if it is fully reprocessed and has not exceeded its maximal allowable storage time in a storage compartment.

Besides the momentarily available endoscopes, such endoscopes can be considered in the availability check, which are currently being in use or undergoing reprocessing, but will be fully reprocessed, and thereby available, at the planned start time of the procedure. Herein, the known durations of examination procedures and separate reprocessing steps may be used for making an appropriate prediction.

The result of the availability check may be displayed to a user of the provisioning system 100 through a user interface 105 displayed on a monitor 104.

Of course, the provisioning system can only make that reliable prediction of when a certain endoscope will be available after reprocessing, if a true description of the respective reprocessing procedure is programmed in the provisioning system. Therefore, when a provisioning system is to be implemented in an application environment, the respective reprocessing procedures can be included in the provisioning system.

While it is of course possible to manually or automatically assess the existing process descriptions retained at the facility, and to translate them into a format readable by the provisioning system, such translation requires significant efforts because the existing process descriptions may not be provided in a machine-readable or standardized data format. At the same time, the process descriptions as retained in the facility may not necessarily accurately describe the reprocessing procedures as actually applied in the facility, due to any later adjustments which may not have been adequately documented in the process descriptions.

For obtaining a more accurate process description with less effort, the provisioning system 100 can be configured to automatically assess log data of previous reprocessing procedures executed in the facility. As such reprocessing procedures must be carefully documented for regulatory reasons, log data can be readily available. Such log data can include, for each procedural step of a reprocessing procedure, an indicator of the type of the procedure, a start time of the procedure, a duration or an end time of the procedure and an identifier of the medical instrument or device undergoing reprocessing. The log data may further include parameters describing the procedural step for example, applied temperatures, a dosage of cleaning or disinfecting chemicals, or the like. The log data can also include an indicator whether or not the procedural step has been completed successfully or not. The log data may further include an identifier of a location or facility in which the reprocessing procedure is being performed.

For analyzing the log data, the provisioning system 100 can comprise a data analyzing engine 200, which may be implemented by a software program running a separate computer, or a software program running on the data processing unit 102. The software may be represented by a sequence of machine readable instructions stored on a machine readable data carrier, for example, a volatile or permanent memory of the separate computer of the data processing unit 102, a hard- or flash disk, or a similar data carrier. For execution of the software program, the machine readable instructions may be read from the memory and subsequently executed by a processor of the separate computer or the data processing unit 102.

The data analyzing engine 200 can be configured to apply various statistical methods for computing accurate process descriptions for different types of medical instruments and devices, such as endoscope types E1, E2, and E3. Such statistical methods include sorting, filtering, averaging, and other known statistical methods.

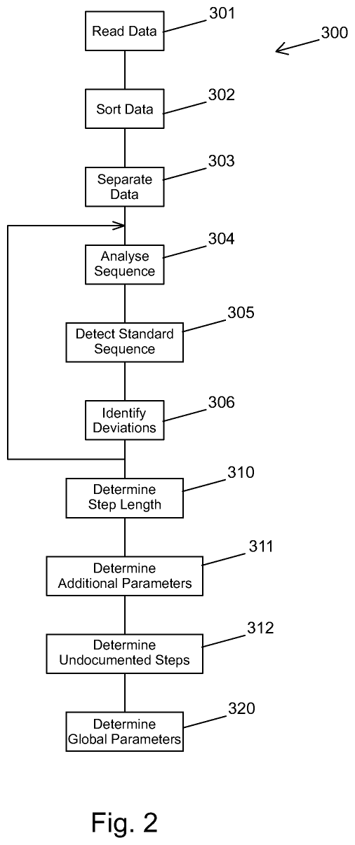

An exemplary algorithm 300 for computing a process description from a body of log data is shown in FIG. 2. Such algorithm can be executed by a processor of the data processing unit 102, as part of a software program implementing the data analyzing engine 200, or by a processor of a separate computer implementing the data an analyzing engine 200. In the following, the algorithm is described in further detail.

In a first step 301, the body of log data is read. The body of log data usually contain several thousand individual data entries, each representing a single procedural step applied in a reprocessing procedure of an individual medical instrument or device.

In a second step 302, log data entries are sorted. In an exemplary embodiment, the log data may be sorted by the type of the instrument or device undergoing treatment as the first criteria, the unique identifier of the instrument or device undergoing treatment as a second criteria, at the start time of the respective procedural step as the third criteria.

In a third step 303, the log data may be separated into several data subsets, each comprising log data of reprocessing procedures of a single type of device or instrument. Each of the so created data subsets can then be separately processed for computing a process description for reprocessing of the respective type of device or instrument.

In step 304, each data subset is analysed to identify the sequence of procedural steps applied during reprocessing of the respective medical device or instrument type. For this, the data indicating the type of the procedural step is used.

Due to the sorting in step 302, the sequence of the data entries in the data subset will directly reflect the sequence of procedural steps applied to an individual medical device or instrument. As each individual medical device or instrument will probably have undergone a plurality of reprocessing procedures, that respective procedures may be isolated by analyzing time gaps between constitutive procedural steps, wherein an unusually long time gap may indicate the end of a reprocessing procedure. In some embodiments, the log data may include log data not only of reprocessing procedures, but also of use of the respective medical instrument or device in an examination procedure. In this case, the completion of a reprocessing procedure can also be recognized in that log data representing a use of the instrument or device directly follows log data representing a procedural step of a reprocessing procedure.

In an embodiment, the data analyzing engine 200 may compile a sequence string for each completed reprocessing procedure, wherein each sequence string comprises an ordered list of data elements representing the type of each procedural step performed in the respective completed reprocessing procedure. In an example, each type of a procedural step may be represented by a short code, for example, a two to four character string. Reprocessing procedures with the same sequence of procedural steps will therefore result in equal sequence strings, while reprocessing procedures with a different sequence of procedural steps will result in a different sequence string.

In step 305, a standard sequence of procedural steps may then be identified. Therefore, the data analyzing engine 200 may count how often a particular sequence string occurs in the data subset. It can be assumed that the sequence string with the highest count represents a standard sequence of procedural steps in a reprocessing procedure for the type of medical device or instrument being subject of the current data subset.

In some embodiments, there may be sequence strings derived from each data subset, which deviate from the standard sequence for the respective data subset. Such deviating sequence strings are used in an optional step 306 for analyzing modifications of the standard reprocessing procedure. By comparing the deviating sequence string with the standard sequence string, the data analyzing engine 200 may determine the kind of deviation. Some examples of deviations are added procedural steps, omitted procedural steps, and loops. A loop may comprise one or more procedural steps which are repeated, before the standard sequence is continued.

Where deviating procedure sequences have been detected, the data analyzing engine 200 may be configured to further analyse log data of procedural steps involved in the deviation, in order to determine probable causes for such deviation. In some embodiments, certain procedural steps may be omitted when specific conditions are met. Some nonlimiting examples may be as follows: A manual precleaning step may be omitted where the reprocessing procedure has been directly preceded by a storage step, which has exceeded a maximum allowable storage time. A channel purge step in a drying cabinet may be omitted if a channel purge step has previously been applied in an endoscope reprocessing machine. A leakage test step in an endoscope reprocessing machine may be omitted, if a manual leakage test step has previously been applied. A pre-rinse step in an endoscope reprocessing machine may be omitted, if a qualified detergent chemical has been used in a previous manual precleaning step. An endoscope drying step may be omitted, if the drying step would have started more than a certain time after completing reprocessing in an endoscope reprocessing machine. An extra rinsing step may be applied in a first reprocessing cycle of an endoscope reprocessing machine following a longer period of inactivity, for example on Monday morning after being an active over the weekend. Loops may generally occur after a previous procedural step has not been completed successfully, which can be identified using the success indicator of respective log data. Such loops are also referred to as fallback loops.

The data analyzing engine 200 may thus compute a complete description of the sequence of procedural steps for a reprocessing procedure of the medical instrument or device type subject of the selected data subset, considering both the standard sequence and possible modifications.

The steps 304, 305, and 306 are repeated for each individual data subset generated in step 303.

In the steps 301 and 302, only the sequence of procedural steps in a reprocessing procedure is analysed, without looking into detailed information of each procedural step. In a next phase of data analysis, certain parameters of individual procedural steps are determined.

In step 310, the duration of procedural step is determined for each type of procedural step. For this, the data analyzing engine 200 may compute an average duration from all data entries representing the respective type of procedural step. In one embodiment, separate average durations may be computed for each type of procedural step, if applied to different types of medical devices or instruments. For example, a manual pre-cleaning step may have a much shorter average duration for a short rigid endoscope having only one instrument channel, than for a flexible endoscope having two or three working channels. In a similar manner, standard deviations of the durations may be computed. From such standard deviations, the data analyzing engine 200 may derive minimum and maximum allowable durations for procedural steps.

Similar to the detection of deviations in step 306, the data analyzing engine 200 may also be configured to detect conditional deviations in the duration of procedural steps. As an example, a manual precleaning step may have a longer standard duration when performed by an operator having less than a required working experience.

Next to the duration of individual procedural steps, further parameters may be determined in optional step 311. Such parameters may be temperatures applied during the procedural step, a type and a dosage of cleaning or disinfecting chemicals applied during the procedural step, or the like. Such data may supplement the process description to be computed so that the process description can later be used as a full process description for regulatory purposes.

Again, similar to the detection of deviations in step 306, the data analyzing engine 200 may further be configured to detect conditional deviations in the additional parameters of certain procedural steps. Such conditional deviations may for example show modifications in a parameter of one procedural step to compensate for deviation in a parameter of the preceding procedural step. By such modifications, it is possible to compensate for an underperformance in one procedural step by providing an over performance in a following procedural step.

In a further optional step 312, the data analyzing engine may analyse the timing information from the log data for identifying any undocumented procedural steps. Such undocumented procedural steps may be identified through gaps between consecutive procedural steps. In many cases, there will be varying gaps between such consecutive procedural steps. A statistical analysis of such gaps can show whether the length of the gaps follows a distribution including durations near zero, or whether there is a “cut-off” duration, under which cases or almost no cases have been observed. It may then be assumed that such minimum duration is representative for an additional procedural step, which has not been documented in the log data. In many cases, such undocumented steps will be transport steps, in which medical devices or instruments are transported from one reprocessing equipment to another reprocessing equipment, for example, from a manual precleaning station to an endoscope reprocessing machine, from an endoscope reprocessing machine to a drying cabinet, or the like. Such undocumented procedural steps may then be included in the process description computed by the data analyzing engine.

In some embodiments, the method according to the present disclosure may be applied to log data acquired in more than one facility, for example, more than one hospital or more than one medical office. For example, a corporate operator of a plurality of hospitals or medical offices may be interested in introducing a planning tool for some or all of the hospitals or medical offices under control of the corporate operator. The plurality of facilities may be located in different places, and may therefore be subject to different global parameters, including environmental conditions for example, average temperatures, humidities, or atmospheric pressures. To account for such different global parameters, it may be necessary to adapt certain parameters of reprocessing procedures.

In a further optional step 320, the data analyzing engine 200 may therefore identify deviations in the durations or other parameters of procedural steps between different facilities. For example, at locations with the higher average humidity, the duration of drying steps may be higher than at locations with lower average humidity. Similarly, pressures applied during a leakage test step may vary according to differences in average atmospheric pressure.

By considering such deviations, it is possible to compute customized descriptions of reprocessing procedures for each facility covered by the log data.

The steps 310, 311, 312, and 320 can be applied using the complete log data, wherein the respective medical instrument or device type can be suitably accounted for in the statistical method applied.

Besides the statistical methods described herein, further statistical methods may be applied in the computing of the process description. Such further statistical methods may involve variance analysis, multiple linear or nonlinear regression, multilevel analysis, structural equation models, or the like.

It will be appreciated that process descriptions computed using the methods described herein provide a high level of accuracy due to the consideration of log data from real procedures. However, as statistical methods applied may have their limits and may only infer probabilities for certain parameters, manual revision of the process descriptions may still be necessary. Still, the efforts necessary to generate the process descriptions can still be significantly reduced by the methods described herein.

Process descriptions computed according to the present disclosure may be stored in a database, and later be used in a planning tool for monitoring availability of medical instruments or devices in a facility, and probably also for regulatory purposes. The process descriptions may also be used for the programming of reprocessing equipment. For example, when a new endoscope reprocessing machine is installed in a facility, the machine may be connected to the database to download the description of those procedural steps which are to be executed by the machine. Thereby, all relevant process parameters can be automatically programmed into the new reprocessing equipment, and the programming efforts can be reduced.

While there has been shown and described what is considered to be preferred embodiments of the invention, it will, of course, be understood that various modifications and changes in form or detail could readily be made without departing from the spirit of the invention. It is therefore intended that the invention be not limited to the exact forms described and illustrated, but should be constructed to cover all modifications that may fall within the scope of the appended claims.

What is claimed is:

1. A method for computing a process description of an automated or semi-automated medical device reprocessing procedure, the process description comprising a plurality of procedural steps including at least one of:
manual pre-cleaning,
automated chemical cleaning and disinfection,
thermal sterilization,
drying, and
storage;
the method comprising:
collecting log data of completed reprocessing procedures, the log data including, for the plurality of procedural steps:
the type of the respective procedural step,
an identifier of a medical device undergoing the procedure,
a start time of the procedural step,
a duration or an end time of the procedural step, and
a success indicator of the procedural step;
feeding the collected log data to a data analyzing engine; and
computing, through the data analyzing engine, a process description of the semi-automated or automated reprocessing procedure,
wherein the process description comprises a sequence of procedural steps necessary for the reprocessing procedure, and the duration of each of the procedural steps.

2. The method of claim 1, further comprising computing, through the data analyzing engine, for at least one procedural step, a process constraint selected from a minimum duration and a maximum duration of the procedural step.

3. The method of claim 1, further comprising identifying, through the data analyzing engine, undocumented procedural steps of a reprocessing procedure, and including such undocumented procedural steps in the process description.

4. The method of claim 1, further comprising identifying, through the data analyzing engine, fallback loops of the reprocessing procedure.

5. The method of claim 1, further comprising identifying, through the data analyzing engine, variations in one procedural step correlating with variations and/or absence of a preceding procedural step.

6. The method of claim 1, wherein the log data comprises log data of reprocessing procedures applied to different types of medical devices, the method further comprising computing, through the data analyzing engine, separate process descriptions for each type of medical device.

7. The method of claim 6, wherein the log data includes a type indicator of the medical device undergoing the procedure.

8. The method of claim 1, wherein the log data includes log data of reprocessing procedures applied at different locations, and the log data includes data identifying the location at which the respective reprocessing procedure has been applied.

9. The method of claim 8, further comprising identifying, through the data analyzing engine, differences between corresponding procedural steps at different locations.

10. A computer program product comprising machine readable instructions for execution by a computer, configured to cause the computer to execute the method claim 1.
